(19) United States
(12) Patent Application Publication
(10) Pub. No.: US 2022/0087694 A1
(43) Pub. Date: Mar. 24, 2022

(54) HYBRID THROMBECTOMY DEVICE AND PROCESS

(71) Applicant: IMCAT, INC., Sacramento, CA (US)

(72) Inventors: Johannes van Vaals, Lakewood Ranch, FL (US); John Kucharczyk, Reno, NV (US); Sheri Albers, Sacramento, CA (US); Gregory Latchaw, Wellington, FL (US); Jacob Coumans, Old Lyme, CT (US); Jacobus Coumans, Old Lyme, CT (US)

(73) Assignee: IMCAT, INC., Sacramento, CA (US)

(21) Appl. No.: 17/027,469

(22) Filed: Sep. 21, 2020

(51) Int. Cl. A61B 17/221

(52) U.S. Cl. CPC A61B 17/221; A61B 2017/22045; A61B 2017/2212

(57) ABSTRACT

The disclosed embodiments generally relate to hybrid thrombectomy devices and processes using such devices. In an exemplary embodiment, the disclosure provides a thrombectomy device to extract a thrombus from a body lumen. The device includes a retraction catheter (150) having a proximal end and a distal end, the retraction catheter configured for insertion into the body lumen; a retracting wire (160) movably positioned inside the retraction catheter, the retraction wire further includes: a plurality of struts (170) coupled to the distal end of the retracting wire at a first end of each of the plurality of struts; a collapsible ring (180) coupled to the second end of each of the plurality of struts; and a collapsible web (190) coupled to the ring and configured to expand into a basket when extended beyond the distal end of the retraction catheter. The collapsible web can be configured to fold into a substantially linear structure to movably fit within the retraction catheter. In addition, an Aligner (1120) structure can be deployed to enforce equidistant position of the struts, preventing the struts, ring and web from collapsing along the side of the clot when retracting the device.

### BACKGROUND

### 1. Field

This disclosure pertains to intravascular medical devices for retracting blood clots from blood vessels in the human body. The same system may be used to remove obstructions from ducts and other cavities of the body, such as, for example, foreign bodies or stones from the urinary or the biliary tracts.

### 2. Background

The present disclosure addresses several challenges with current thrombectomy procedures in high risk deep vein thrombosis (DVT) and pulmonary embolism (PE) populations, as well as ischemic stroke (IS) patients.

The present disclosure may be able to capture both hard and soft clots, it may be able to collect large clots in a single pass, rather than relying upon the lengthy and uncontrolled infusion of thrombolytic drugs with potential side effects, it may do so without trauma to the vessel or duct, it may significantly reduce blood loss which can be substantial with other devices, it may be able to reduce or completely eliminate the potential for distal embolization by capturing all of the clot for retraction and preventing release of clot fragments distal to the clot, the catheter system may be substantial smaller making vascular access easier and more rapid, and it may be significantly simpler and more rapid to operate than existing thrombectomy devices.

### SUMMARY

The present disclosure describes a medical device capable of retracting thrombus or another obstacle from blood vessels or other lumens. According to the disclosure, the thrombectomy device retracts the thrombus towards the catheter. It can optionally be equipped with a collecting mechanism and/or aspiration. Once the clot has been drawn into the guiding catheter, the retraction device and clot are withdrawn proximally through the guiding catheter out of the body.

One aspect of the present disclosure is to provide a mechanical thrombectomy system that is small and flexible enough that it can reliably and safely navigate tortuous blood vessels to the site of a thrombus.

A second aspect of the present disclosure is to provide a mechanical thrombectomy device that can reliably and securely entrap a soft or hard thrombus without fragmenting the thrombus or damaging the intima of the blood vessel.

A third aspect of this disclosure is to provide a mechanical thrombectomy device that is biocompatible and compatible with standard medical catheters.

A fourth aspect of this disclosure is to provide a mechanical thrombectomy device that is visible on X-ray, and/or MR imaging, and/or Ultrasound. In addition, fiber optic technology (FOSS, Fiber Optic Shape Sensing) can be embedded to support 3D visualization of the shape and location of the device without any external imaging.

A fifth aspect of the present disclosure is to integrate hemo-compatible materials to improve catheter tip navigability and vascular access.

A sixth aspect of the disclosure is to provide a mechanical thrombectomy device that reduces the risk of fragmentation and distal embolization.

A seventh aspect of the disclosure is to provide aspiration through the guiding catheter, and also through the collecting catheter if it is used to decrease clot fragment embolization, to remove soft components of the thrombus, and to decrease the size of the retracted thrombus to facilitate its removal through the guiding catheter.

An eighth aspect of this disclosure is that the thrombectomy device is pre-loaded within the retraction catheter for easy use.

A ninth aspect of this disclosure is that the retraction catheter has multiple lumens, including one for a pre-loaded thrombectomy device and another one for a guidewire.

### BRIEF DESCRIPTION OF THE DRAWINGS

The disclosure will be described in relation to the following exemplary and non-exclusive illustrations in which similar elements are numbered similarly, and where:

FIGS. 1a-1g show steps in a an exemplary thrombectomy procedure according to certain disclosed principles;

FIGS. 2a-2b are schematic side views of the device for capturing thrombotic material in lumens according to certain disclosed principles;

FIG. 3 is a schematic view of struts and ring segments according to one embodiment of the disclosure;

FIGS. 4a-4c are schematic side views of the device for capturing objects, with one pull/push wire shaped in the form of a tube (web not drawn);

FIG. 5 is a schematic side view of struts and ring segments with asymmetric position of opposing connection points of ring segments (web not drawn);

FIGS. 6a and 6b are a schematic view with multiple struts according to one embodiment of the disclosure;

FIG. 7 is a schematic of the handle according to one embodiment of the disclosure;

FIGS. 8a, 8b, 8c and 8d are schematic representation of the so-called butterfly embodiment according to one embodiment of the disclosure;

FIGS. 9a-9d show schematic views of the butterfly concept with additional pull/push wire (web not drawn);

FIG. 10 is a schematic view of the device with embedded fiber-optics according to one implementation of the disclosure; and

FIGS. 11a-11c schematically represent deployment of an exemplary device with an Aligner according to one embodiment of the disclosure.

The drawings are exemplary and non-limiting and intended to cover modifications, equivalents, and alternatives covered within the scope of the disclosure.

### DETAILED DESCRIPTION

The thrombectomy devices disclosed in this disclosure generally use a web structure with the aid of retraction wires to reliably retract thrombus and other obstructions in blood vessels and body ducts. The device includes expandable struts having a closed compact configuration and an open expanded configuration. In optional embodiments, additional functionality may be optionally included.

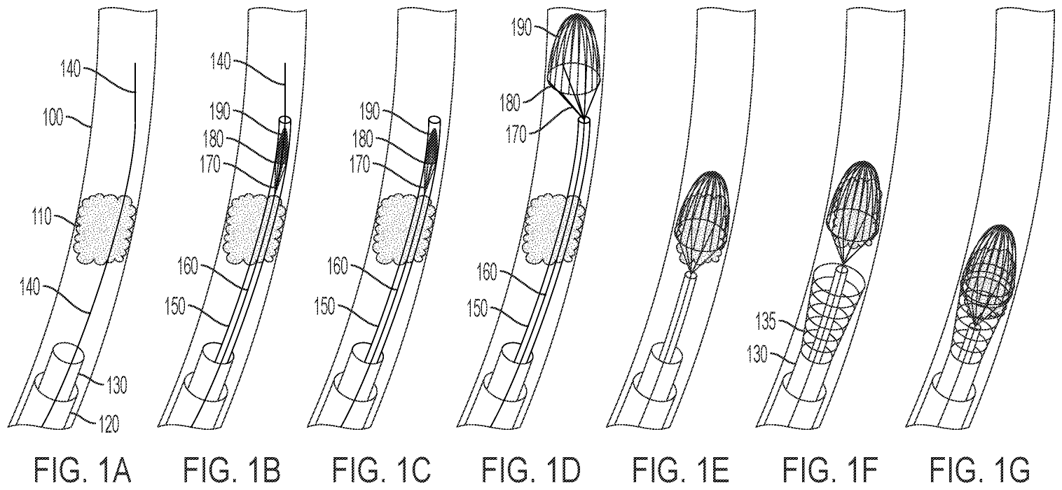

FIGS. 1a-1g schematically illustrate an exemplary embodiment according to one embodiment of the disclosure. Specifically, FIGS. 1a-1g illustrate steps in a an exemplary thrombectomy procedure according to certain disclosed principles. The embodiments of FIGS. 1a-1g depict a workflow with the disclosed device for mechanical thrombectomy device in vessel 100 with a thrombus or clot 110. Guiding catheter 120 may be positioned by image-guided percutaneous transluminal catheter delivery within the lumen of the blood vessel 100 proximal to the thrombus, using angiography, ultrasound, MRI, or by Fiber Optic Shape Sensing (FOSS) techniques.

A collecting catheter 130 may be optionally included. Collecting catheter 130 may pass through guiding catheter 120 and may be placed just below the proximal aspect of clot 110. Collection catheter 130 and guiding catheter 120 may be concentric or non-concentric. In one embodiment, guidewire 140 can be placed distal to thrombus 110 as shown in FIG. 1A and may be used to guide retraction catheter 150. Retraction catheter 150 can then pass through collecting catheter 130 and over guidewire 140 to a position with its distal tip placed distal to the distal edge of the thrombus 110. This is shown at FIG. 1B where retraction catheter 150 containing guidewire 140 is extended through thrombus 110.

In one embodiment, the thrombectomy device with retracting wire 160, struts 170, ring structure 180 and web 190 may extend, and pass through, retraction catheter 150. In another embodiment, retracting wire 160, struts 170, ring structure 180 and web 190 may be pre-loaded inside the retraction catheter. Guidewire 140 can be removed, as shown in FIG. 1c, where the thrombectomy device is shown collapsed inside retraction catheter 150.

The thrombectomy device (including components 170, 180, 190) can be deployed by manipulating the retraction wire 160 as shown in FIG. 1d. Retraction catheter 150 can be pulled proximally with the thrombectomy device pulled down over thrombus 110, as shown in FIG. 1e. Optionally, as shown in FIG. 1f, collecting basket 135 may be deployed from the collecting catheter 130 to collect the thrombus, as shown in FIG. 1g. In certain embodiments, Aspiration may be also applied to the guiding or collecting catheters to decrease embolization of clot fragments and to aid in the removal of the thrombus.

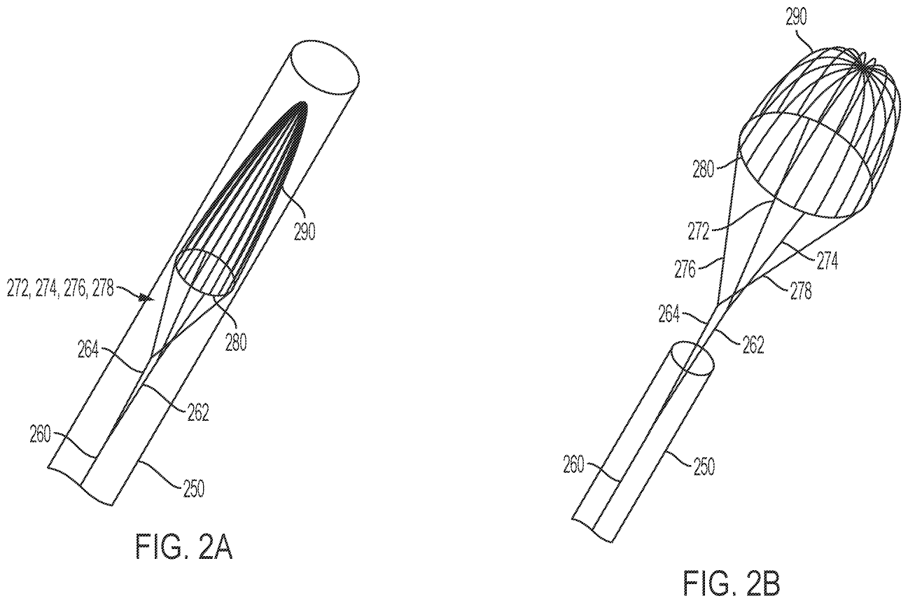

FIGS. 2a-2b are schematic side views of the device for capturing thrombotic material in lumens according to certain disclosed principles. Specifically, FIGS. 2a and 2b illustrate retraction catheter 250 having retraction wire 260 with struts 272, 274, 276 and 278 optionally attached to ring-like configuration 280 and connected to the retraction wires 262 and 264. In one embodiment the retraction wires 262 and 264 may merge into a single retraction wire 260, or in another embodiment the struts may directly merge into a single retraction wire 260. In a further embodiment, the number of struts is not limited to 4, and the number of retraction wires is not limited to 2 but can be increased or decreased to accommodate the desired application. In one embodiment, finely woven web wires 290 extend from the ring for a variable distance. Web wires 290 may be comprised of platinum or nitinol, thereby allowing components to be packed into the catheter and which further allow pressurized heparinized saline to percolate between the components.

Initially, as shown in FIG. 2a, the device is collapsed inside the retraction catheter 250. Next, as shown in FIG. 2b, the device is deployed and ring 280, which in one embodiment approximately covers the vessel lumen, is positioned and gently pulled proximally with retraction wire 260 over the thrombus while the web is entirely covering the thrombus.

Similar systems disclosed in the present disclosure may preferably be used to remove obstructions from ducts and other cavities of the body, such as, for example, bile or pancreatic ducts, or another foreign body. In an exemplary embodiment, a collecting basket and catheter may be used to capture such clot or object. According to an embodiment of the disclosure, any obstruction, retraction wire, and retraction catheter can be pulled into the collecting catheter or directly into the guiding catheter for removal.

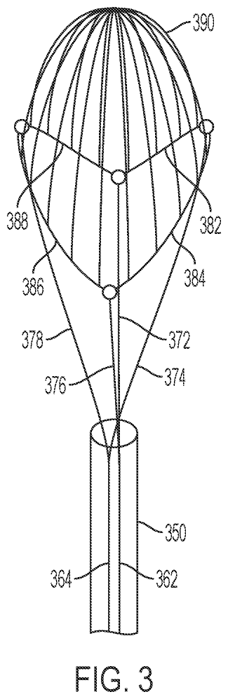

FIG. 3 is a schematic view of struts and ring segments according to one embodiment of the disclosure. As shown in FIG. 3, the thrombectomy device can be used to pull a thrombus from a blood vessel with retraction wires 362 and 364 passing through a retraction catheter 350. In one embodiment, retraction wires 362 and 364 extend into struts 372, 374, 376 and 378 which may be expandable, flexible and freely movable. In one embodiment, curved ring sections 382, 384, 386 and 388 are connected via struts 372, 374, 376 and 378, which can merge into individual retraction wires 362 and 364. Ring segments 382, 384, 386 and 388 may be flexible and can be aligned inside the retraction catheter before deployment. Once deployed, struts 372, 374, 376 and 378 as well as ring segments 382, 384, 386 and 388 expand to proximate a ring inside the vessel lumen. In some embodiment, web 390 can be coupled to the ring structure and deployed to engulf, capture or otherwise surround a clot or an object. In a further embodiment, the number of struts and ring segments is not limited to 4, and the number of retraction wires is not limited to 2 but can be increased or decreased to accommodate the desired application.

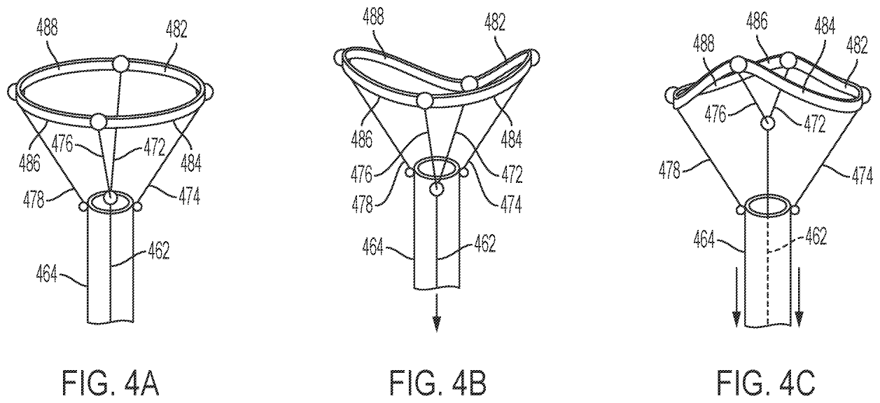

FIGS. 4a-4c are schematic side views of the device for capturing objects, with one retraction wire shaped in the form of a tube. More specifically, the embodiments of FIGS. 4a and 4b show retraction tube 464 housing retraction wire 462. For simplicity, retraction catheter (350 in FIG. 3), and the web (390 in FIG. 3) are not shown. Struts 472 and 476 are connected to the connection between ring section 482 and 488, and 484 and 486, respectively, and merge into retraction wire 462. Struts 474 and 478 are connected to the connection between ring section 482 and 484, and 486 and 488, respectively, and merge into retraction tube 464.

FIG. 4b shows one implementation of the disclosed principles in which retraction wire 462 is moved in a first direction relative to retraction tube 464. As illustrated, the movement causes extension of struts 474 and 478 relative to struts 472 and 476, causing collapse of the ring formed by ring segments 482, 484, 486 and 488.

FIG. 4c is another implementation of the disclosed principles in which retraction tube 464 is moved in a second direction relative to retraction wire 462. As illustrated, the movement causes extension of struts 472 and 476, which thereby extends ring segments 482, 484, 486 and 488 to form a convex protrusion.

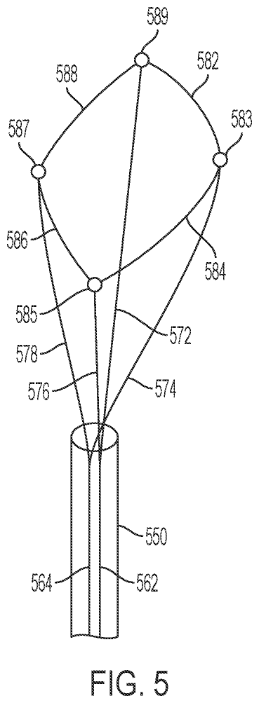

FIG. 5 is a schematic side view of struts and ring segments with asymmetric position of opposing connection points of ring segments. The connection points 583, 585, 587 and 589 are connecting ring segments 582 with 584, 584 with 586, 586 with 588, and 588 with 582, respectively. In FIG. 5, opposing retraction wires 562 and 564 merging into struts 572 and 576, and 574 and 578, respectively, and attached to points 585 and 589, and 583 and 587, respectively, can be moved to different relative positions with one positioned more proximal and the other more distal. This facilitates folding the device with the ring structures in such a way that when folded together to fit inside the retraction catheter 550, segments 582 and 588 are parallel but distal to the parallel segments 584 and 586 and will allow a folded position that has a smaller footprint radially. In FIG. 5, the web (390 in FIG. 3) is not shown.

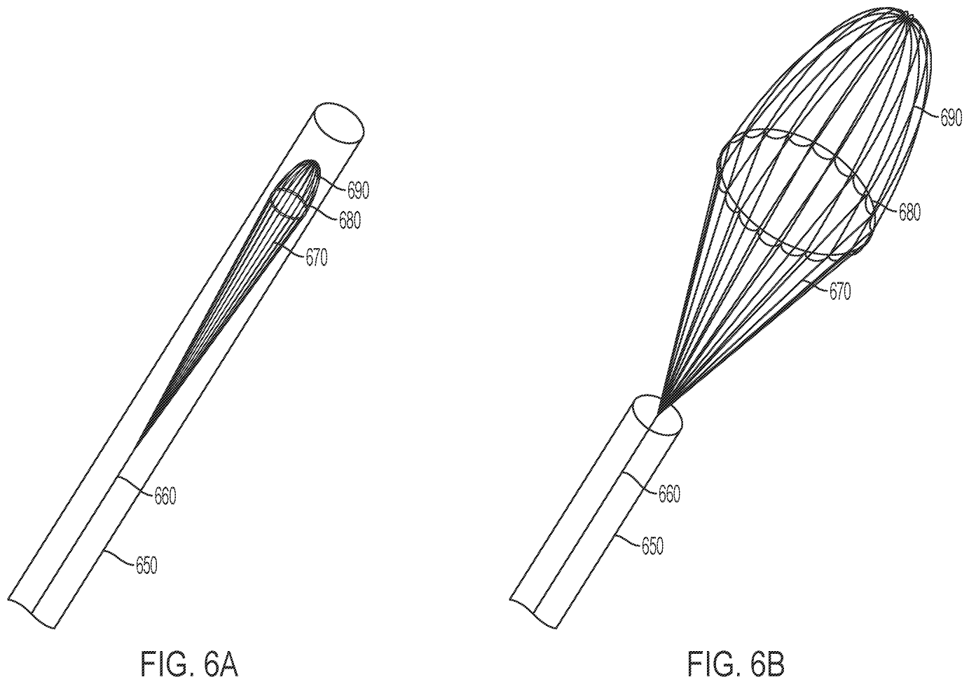

FIG. 6 is a schematic view with multiple struts according to one embodiment of the disclosure. In FIG. 6a, multiple struts 670 extend from extraction wire 660 contained within the extraction catheter 650. The struts, or the connected web wires 690, may be spring-loaded or made of memory material with bias to deploy towards the lumen of the vessel. The web 680 may be coupled to the multiple struts 670.

FIG. 6a shows a pre-loaded device inside extraction catheter 650. FIG. 6b shows the retraction device deployed. Ring-shape 680 may thus be formed by individual struts 670 operating independently to shape web 690 into a ring-like opening. Alternatively, web 690 may assume a predefined shape when deployed due to its inherent bias. In still another embodiment, ring-shape 680 may operate in the manner of spring-loaded struts 670 that open like an umbrella to radially expand towards the wall of the blood vessel. Radial expansion is typically only limited by the lumen diameter of the blood vessel. In one embodiment, ring-segments can connect the end of the struts, and in another embodiment instead of ring-segments connecting the ends of the struts, the ends of the struts may be connected with ring wires which can be flexible. In one embodiment, the web 690 can be connected to the plurality of ring wires, and in another embodiment, the web can be attached to the ends of struts 670.

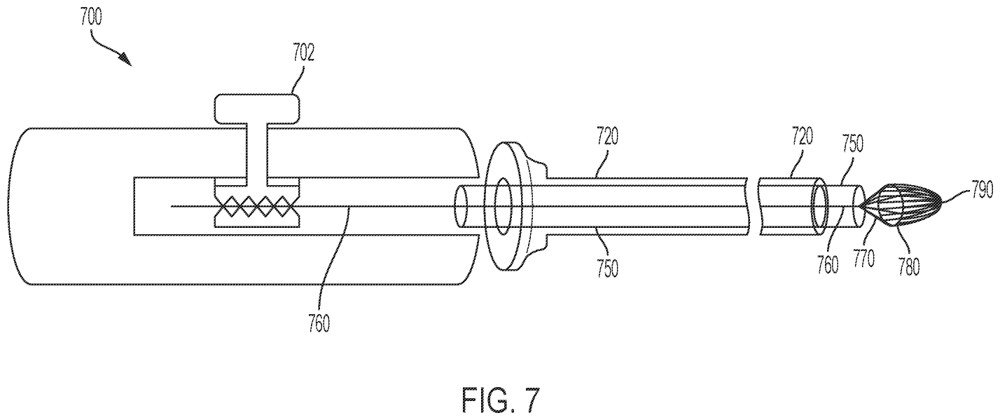

FIG. 7 is a schematic of the handle according to one embodiment of the disclosure. As shown in the embodiment of FIG. 7, an exemplary hybrid thrombectomy device may be equipped with handle 700 attached to retraction wires 760 and/or retraction catheter 750, and/or the guiding catheter 720. The disclosed embodiment allows the manipulation of one or more retraction wire(s) 760 for the deployment and retraction of the thrombectomy device (consisting of the struts 770, ring-shape 780, and web 790) by one or more controls 702 on the device handle. In another embodiment, additional controls may be included to control other elements such as the optional collecting catheter and basket (not shown) and aspiration. In an embodiment with FOSS technology the handle can incorporate connecting the optical fibers embedded in the catheters and/or wires, and in another embodiment the handle may incorporate the laser and other components that are needed for FOSS.

Handle 700 may include a deployment and/or retraction mechanism as well as notches, indents, bumps, protrusions, and other features. The handle may also have alternative shapes or forms made out of a polymer material, a metal material, a combination of a metal material and a polymer material, or more other suitable materials. In the method of the disclosure, the thrombus, ring, and web are pulled proximally into the collecting catheter, which is then pulled out of the guiding catheter.

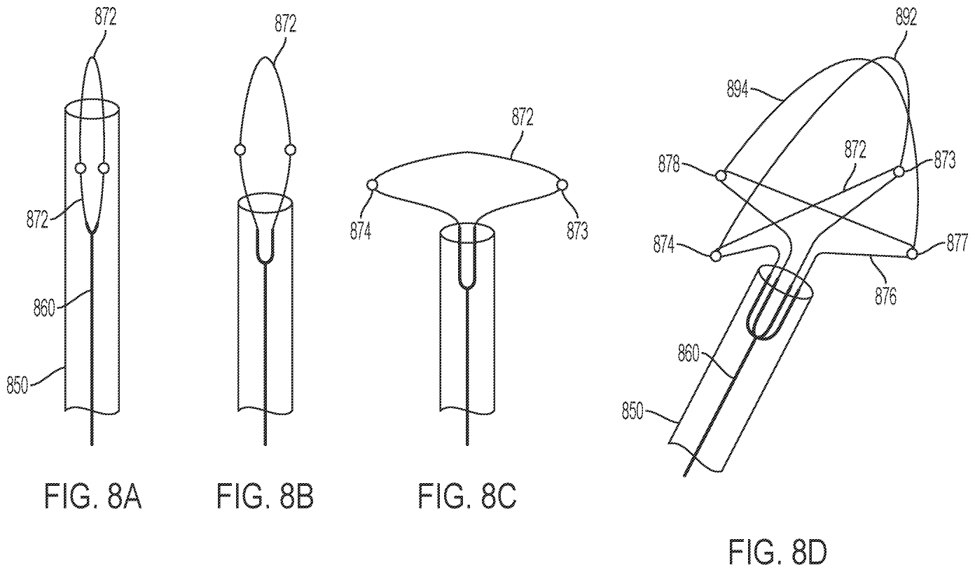

FIGS. 8a, 8b, 8c and 8d are schematic representations of the so-called butterfly embodiment according to one embodiment of the disclosure. Here, retraction catheter 850 is preferably configured with a retraction wire 860 extending into the first butterfly ring 872 and second butterfly ring 876, and first web wires 892 and 894, respectively. Web wires 892 and 894 are coupled to the outward points of the butterflies 873/874 and 877/878, respectively. A plurality of web wires can be coupled to other web wires that complete the butterfly ring-shaped base for the web. In a further embodiment, the number of butterfly structures can be any number including the two depicted in FIG. 8d. FIGS. 8a, 8b and 8c only show one butterfly and do not show the web wires. In FIG. 8d, only a few web wires (892 and 894) are drawn, and the complete web is not drawn for clarity.

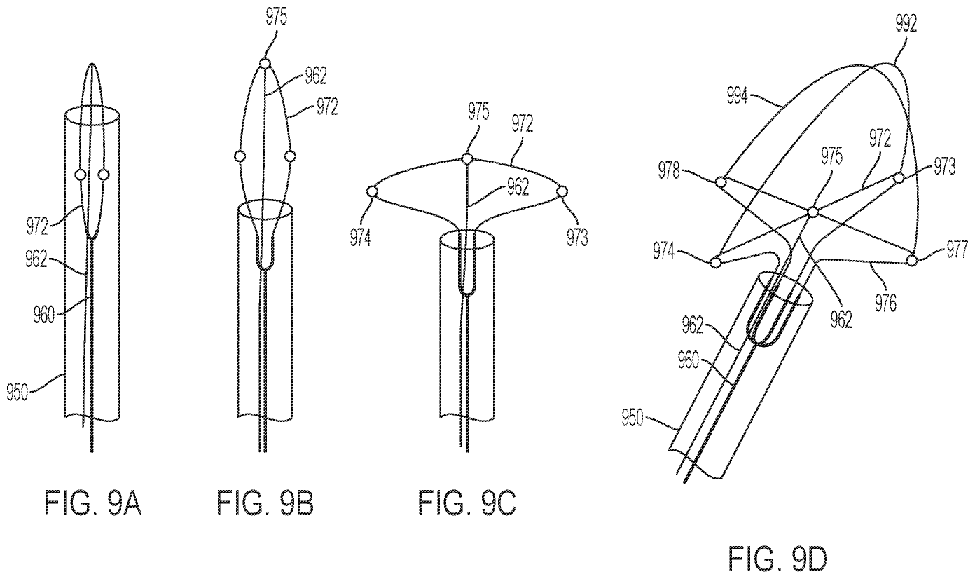

FIGS. 9a-9d show schematic views of the butterfly concept with a pull/push wire in addition to the retraction wire. Specifically, the embodiments of FIGS. 9a-9d show an additional retraction wire 962 connected to apex 975 of the intersecting butterflies. Retraction wire 962 assists the deployment of the butterfly structures 972 and 976 by pulling back apex 975 of the butterfly structures after the complete butterflies have been released from the retraction catheter. Once in the deployed state, retraction wire 962 can be pushed out to assist the return to the ‘stretched-out’ configuration of the butterfly structures so that the structure can be retracted back into the retraction catheter. Once again, the complete web is not drawn for clarity. As in FIG. 8, a plurality of web wires 992 and 994 and more are connected to the butterfly structures to create the web that captures the thrombus.

In one embodiment of the disclosure, the thrombectomy device including the retraction wires, struts, ring-shape and web can be pre-loaded in the retraction catheter. One advantage of having the thrombectomy device pre-loaded is that it is easier for the clinical user: there is no need to pass it through the retraction catheter, and since it can be pre-loaded at a well-defined position at the distal tip of the retraction catheter, and with the retraction catheter moved to the position distal to the thrombus, the only manipulation needed to deploy the actual thrombectomy device is to push the retraction wire(s) over a well-defined distance to deploy the thrombectomy device. This distance can be pre-set in the handle, thereby making it very reliable and user friendly, requiring minimal extra skills of the clinical user.

In another embodiment, to secure easy transfer of a guidewire through the retraction catheter without the need to physically pass next to the thrombectomy device, the retraction catheter can be equipped with multiple lumens (each of independent size as required), where one lumen can be used for the thrombectomy device (preferably ‘pre-loaded’), and another lumen can be used for a guidewire.

In another embodiment, a catheter connected to an aspiration device can be added to the manifold attached to the hub of the guiding catheter or if present the collecting catheter so that aspiration is applied to the entire system to facilitate retraction, prevent fragmentation and embolization of firm fragments, and aspiration of softer elements to facilitate clot removal through the guiding catheter.

In another embodiment, the device can be equipped with imaging sensors, or with sensors measuring physiological parameters such as pressure, temperature, oximetry.

In another embodiment, a thrombolytic or therapeutic agent can be released as part of the procedure prior, and/or during/and or immediately following the thrombectomy procedure. The agent can be released via one of the catheters that are in use for the thrombectomy procedure, or via an additional catheter passed through the guiding or collecting catheter.

In one embodiment of the disclosure, fluoroscopically visible markers are applied to the retraction ring, to the retraction wire and the collecting basket and to the tip of the guiding catheter, the collecting catheter and the retraction catheter to facilitate localization of all components.

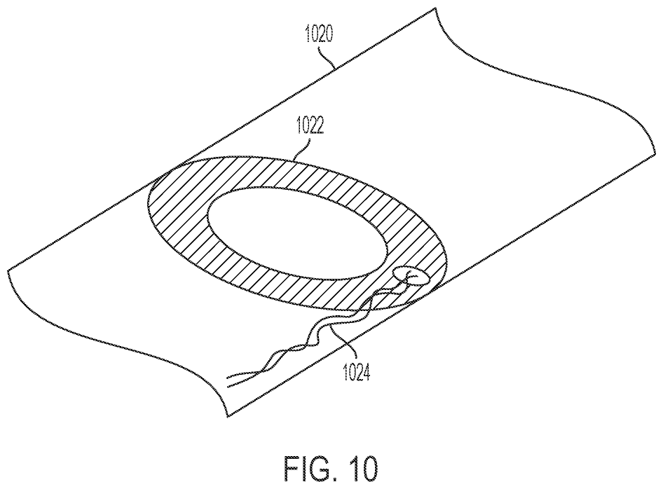

FIG. 10 is a schematic view of the device with fiber-optics 1024 embedded in the wall 1022 of a catheter 1020, according to one implementation of the disclosure. In FIG. 10, Fiber Optic Shape Sensing (FOSS) device includes optical fibers 1024 which can be applied to or embedded in all components, such as any of the catheters and wires used, so that fluoroscopy can be decreased or eliminated for improved safety for the patient and the medical staff conducting the procedure.

In one embodiment, FOSS technology enables the manipulation and visualization of thrombectomy devices without the need for fluoroscopy. In addition to reducing the need for x-ray exposure of the patient and medical personnel, the FOSS technology also features improved, more accurate, easier and faster guidance by providing more detailed views of device positioning with 3D views that are difficult to achieve with fluoroscopy. In an exemplary embodiment, optical fibers are embedded in the catheters or other parts of the device, with typically 3 or more micro-optical fibers that are equipped with Fiber Bragg Gratings. This enables (by analyzing the reflected laser light coupled to the fibers) the determination in 3 dimensions of the shape and position of the catheters and wires in real-time and with high accuracy. The shape and position of the catheters and wires can then be superimposed on roadmap views of the vasculature and pathology.

Portions or the components of a thrombectomy device may also preferably include a radiopaque material capable of producing a relatively bright image on a fluoroscopy screen, or another imaging technique during a medical procedure which aids the user of the retraction thrombectomy device in determining its location. Some examples of radiopaque materials can include, but are not limited to, gold, platinum, palladium, tantalum, tungsten alloy, and polymer material loaded with a radiopaque filler. Components of the thrombectomy device may also be made from a metal, metal alloy, polymer, a metal-polymer composite, ceramics, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys.

According to the present disclosure, FOSS can be used to reduce or eliminate X-ray utilization and to improve guidance and visualization. In shown in FIG. 10, fiber optics can be embedded into one or more elements of the device, for example, the guidewire, catheters, struts and wires. Reflected laser light applied to the fibers can be used to calculate in 3D the shape of the device. The 3D views of the shape can be superimposed in real-time on roadmaps (obtained with X-ray, MR or US), that can be rotated to create visualization from any direction, all without the need for additional fluoroscopy. The benefits of FOSS include improved visualization, easier guidance, faster procedures, and reduced or no radiation. In the method of the disclosure, roadmap and pathology visualization may be done with US combined with device guidance performed using FOSS, which would provide a cheaper and more accessible therapeutic solution.

When using two devices like a FOSS guidewire running through a FOSS catheter, there is an obvious space constraint where the guidewire runs inside the catheter. The FOSS data for guidewire and catheter may be aligned with each other in post processing in order to increase spatial precision—or when the discrepancy between the calculated positions is too large, that may be used as an indication that the shape of either has a higher inaccuracy. Monitoring the distribution shape of flexible components, FOSS can provide valuable data during design, testing and operation, including potentially in vivo vascular conditions.

The thrombectomy device product may be provided in any format of packaging that protects the device and preferably keeps the device in antiseptic conditions for single sterile use. In one embodiment, a rigid polyethylene tube carrier may be inserted into a Tyvek/polyethylene pouch pre-sealed on three sides. The pouch can be heat-sealed closed with a label placed on the clear polyethylene film. A sealed pouch along with Instructions for Use may be inserted into an appropriately sized carton.

The wires used to practice the present disclosure may be produced from any number of suitable materials. Preferably, the wire may be made from a “so-called” super-elastic alloy. These alloys are characterized by an ability to transform from an austenitic crystal structure to a stress-induced martensitic structure and to return elastically to the austenitic crystal structure (and the original shape) when the stress is removed. A typical alloy is nitinol, a nickel-titanium alloy, which is commercially available and undergoes the austenite-SIM-austenite transformation at a variety of temperature ranges. In the method of this disclosure, the thrombectomy device is attached to a nitinol retraction wire and sheathed by a flexible retraction catheter. A colored positioning marker on the retraction catheter aids in proper insert placement. A handle can control the device delivery and release mechanism. The thumbwheel on the handle retracts the retraction catheter. The button allows the physician to change the function of the thumbwheel from retracting the retraction catheter to deploying the device. The introducer helps facilitate entry and advancement of the insert during insertion of the device. The retraction wire is detached from the device by continuing to rotate the thumbwheel. Symbols are located on the handle.

In an exemplary practice according to the disclosed principles, a method of imaging, including ultrasonography, computed tomographic angiography, or magnetic resonance angiography can be used to localize thrombus. Puncture of the right femoral vein located just inferior to the inguinal ligament can be performed and a standard large bore (16F or larger) intravascular sheath connected to heparinized saline can be introduced into the vein. A 16F 65 cm long guiding catheter connected to heparinized saline containing a standard 0.035″ J-tip guidewire can be introduced into the sheath. The J-tip guidewire can be pushed cephalad to just below the thrombus. The guiding catheter can be pushed cephalad, sliding smoothly along the J-wire so that its tip may be approximately 4 cm proximal to the thrombus. A 14F catheter with a 4 cm long compressed nitinol basket (the collecting device) at its distal end can be placed over the J-wire and advanced so that the basket may pass out of the distal end of the guiding catheter, opening to fill the entire lumen of the vein proximal to the thrombus. The J-guidewire is removed and both catheter systems are aspirated and then flushed with heparinized saline.

A 6F catheter may be employed with a stainless steel 0.025″ guidewire with a floppy distal end and can be passed cephalad into the collecting catheter so that its tip extends into the venous lumen, the boundaries of which are lined with the collecting system nitinol basket. In one embodiment, a small guidewire can be pushed cephalad so that it may pass between the clot and the intimal surface of the vein. The retraction catheter can then be pushed cephalad to follow over the guidewire so that its tip ends distal to (above) the thrombus. The guidewire may be exchanged for the retraction device, or the retraction device may already be present pre-loaded, preferably in another lumen within the retraction catheter.

In the present disclosure, the retraction catheter and retraction wire may pull the clot into the collecting catheter, which closes tightly over the thrombus as it is pulled into the guiding catheter. When possible, the entire thrombus may be pulled into the guiding catheter and removed from the body, leaving the guiding catheter in place. If the clot is too large to be pulled into and through the guiding catheter, aspiration may be applied to the guiding catheter or the collecting catheter, wherein in an embodiment of the disclosure, a catheter connected to an aspiration system can be hooked to the flushing system for the guiding catheter via a 3-way stopcock. Aspiration can be usefully added when applied to the clot that has been pulled into the collecting device to make it smaller for removal through the guiding catheter. In a further embodiment of the disclosure, J-wires with very firm distal ends may be preferably introduced into the guiding and collecting catheters to break the clot into smaller pieces while continuous aspiration prevents the clot fragments from flowing distally. If not successful, the clot adherent to the tip of the guiding catheter, and the catheter itself, can be removed through the access sheath. Catheterization with the guiding catheter can then be repeated, as needed.

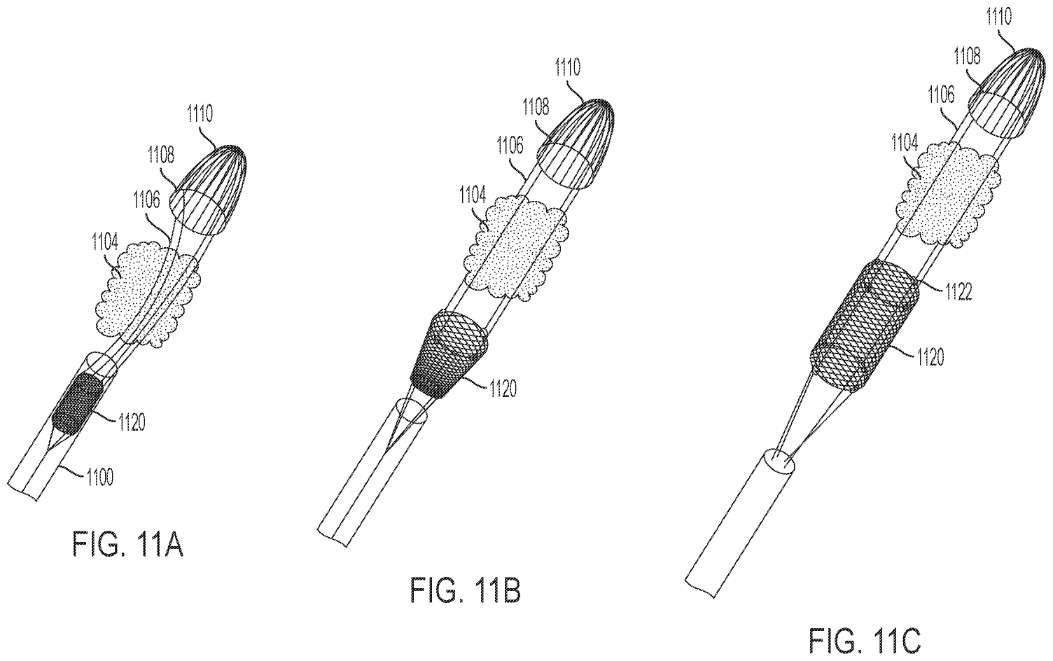

A problem often encountered during the deployment and retraction of conventional thrombectomy devices is that the device slides to one side when encountering the clot. Thus, the device is not pulled over the clot; rather, it passes in-between the clot and the vessel wall. An exemplary embodiment of the disclosure addresses this shortcoming by incorporating a so-called Aligner into the thrombectomy device. FIGS. 11a-11c schematically illustrate one such embodiment and its application.

Specifically, FIG. 11a shows exemplary device 1100 positioned such that ring 1108 and web 1110 are distal to clot 1104, but with the Aligner 1120 proximal to clot 1104. In FIG. 11a, the Aligner 1120 has not yet deployed and is still within the retraction catheter 1100. In FIG. 11a the device is halfway deployed; that is, web 1110 and ring 1108 are fully deployed. The retraction catheter is withdrawn below clot 1104 but the Aligner 1120 is still within the extraction catheter.

The struts 1106 may pass the clot mostly on one side since that is typically the path created by the guidewire (not shown) and followed by the withdrawn extraction catheter (not shown). As a result, the problem arises in that struts 1106 will not evenly distribute over the circumference of the vessel lumen (not shown) and will typically cluster on one side of the clot 1104. This may not be a problem for soft clots assuming that when withdrawn, struts 1106 will probably cut through the clot. With the ring not collapsing, clot 1104 would be captured.

In the case of a hard clot, however, the struts cannot cut through the clot when pulled. Thus, ring 1108 and web 1110 will collapse following the path of the struts thereby passing clot 1104 on a side and leaving the clot behind.

FIG. 11b shows how this can be prevented using the disclosed embodiments. Specifically, FIG. 11b shows the next step with the extraction catheter further withdrawn using the Aligner 1120. The release of the Aligner 1120 forces struts 1106 to move away from each other towards the four quadrants of the vessel circumference (for example, at the 0°, 90°, 180°, and 270° positions). Struts 1106 are forced to these positions upon deployment of Aligner 1120, since they are passing through the Aligner 1120 at the 0°, 90°, 180° and 270° positions respectively. In this manner, the Aligner 1120 acts as to reinforce the struts 1106, keeping them open throughout the retraction phase. This is achieved by weaving struts 1106 through the Aligner structure or by having eyelets 1122 or similar structural features to fix the passing struts (indicated with circles in FIG. 11b) at the designated positions. At least some of the struts 1106 can still freely move up and down to facilitate manipulation of the ring segments. Optionally, the struts 1106 can be enforced to relocate from passing from one side alongside the clot to equidistant positions surrounding the clot by rotating the Aligner.

FIG. 11c illustrates the fully deployed device. Here, device 1100 can be retracted without the risk of collapsing of the ring, enabling full enclosure of the clot by the ring and web.

Another practical issue addressed in the present disclosure is the accuracy of the navigational process used to direct the endovascular placement of a thrombectomy device relative to the location of a thrombus. Magnetic Resonance Imaging (“MRI”) can help localize and characterize the thrombus and optimize the positioning of the thrombectomy device. In one embodiment of the disclosure, high-speed, high-resolution MR imaging is combined with conventional X-ray fluoroscopy and digital subtraction angiography (DSA) capability in a single hybrid imaging unit. This real-time imaging capability makes it possible to use high-speed MR imaging to direct the movement of catheters and other components of the thrombectomy system to specific endovascular locations, and thereafter observe the effects of specific interventional procedures.

Magnetic Resonance Imaging. After acquiring a roadmap visualizing the vasculature and pathology, it is possible to project the position of the device in 3D. Advancing the catheter toward the thrombus and manipulating the device while extracting the thrombus can be done without the need for any additional x-ray, with better and more accurate 3D visualization, under all projections, and typically in a shorter time. The catheter tip on thrombectomy devices is difficult to see on MRI because of inadequate contrast with respect to surrounding tissues and structures. This makes accurate localization difficult and degrades the quality of the diagnostic information obtained from the image. Thus, one objective of this disclosure is to provide an MR-compatible and visible device that significantly improves the efficacy and safety of thrombus removal using MR guidance. For example, to enhance compatibility with MRI imaging systems, it may be desirable to make portions of the device in a manner that would impart a degree of MRI compatibility. For example, the device, or portions thereof, may be made of a material that does not substantially distort the image and create substantial artifacts (artifacts are gaps in the image). Certain ferromagnetic materials, for example, are not preferred in the present disclosure because they may create artifacts in an MRI image; rather, the device may be made from a material that the MRI machine can image.

Different elements of the device, including elements such as ring segments or alternatively connective eyelets of ring segments can be made visible on MRI by selecting a coating of, a core of, or an amalgam of nitinol, platinum and ferromagnetic material. Circular structures such as the ring shape can be configured in such a way that they create resonating structures excited and made visible by parts of the MRI imaging sequence.

MR imaging may also be of practical benefit in the present disclosure in assessing and in characterizing the age, composition, and size of a thrombus. A growing body of evidence suggests that a combination of MR imaging and neurologic symptoms may in fact have prognostic predictive value in assessing patient outcome. During formation of a thrombus, the blood contains a mixture of oxyhemoglobin, deoxyhemoglobin and methemoglobin that is usually equal to that of arterial blood. As the thrombus ages, however, the concentration of paramagnetic hemoglobin and methemoglobin within the clot also changes resulting in a characteristic appearance on MR images that reflects the age and stability of the clot. Observation of these MR imaging changes can be clinically useful in the method of the present disclosure in evaluating the potential utility of various alternative interventions, such as, for example, drug thrombolytic therapy and mechanical thrombectomy.

Further in the method of the disclosure, materials can be added to the structure of a pliable catheter to make it MR visible but may not contribute significantly to the overall magnetic susceptibility of the catheter, or imaging artifacts could be introduced during the MR process. In one embodiment, thrombectomy devices used under MR guidance are MR-compatible in both static and time-varying magnetic fields. Although the mechanical effects of the magnetic field on ferromagnetic devices present the greatest danger to patients through possible unintended movement of the devices, tissue and device heating may also result from radio-frequency power deposition in electrically conductive material located within the imaging volume. Consequently, in the method of the present disclosure, all cables, wires, and devices positioned within the MR imaging system must be made of materials that have properties that make them compatible with their use in human tissues during MR imaging procedures. Many materials with acceptable MR-compatibility, such as ceramics, composites and thermoplastic polymers, are electrical insulators and do not produce artifacts or safety hazards associated with applied electric fields. Some metallic materials, such as copper, brass, magnesium and aluminum are also generally MR-compatible. Guidewires for the catheter component of the thrombectomy system can usually made of radiopaque material so that their precise location can be identified during a surgical procedure through fluoroscopic viewing.

The following non-limiting examples are provided to further illustrate some embodiments of the disclosed principles. Example 1 is directed to a thrombectomy device to extract a thrombus from a body lumen, the device comprising: a retraction catheter (150) having a proximal end and a distal end, the retraction catheter configured for insertion into the body lumen; a retracting wire (160) movably positioned inside the retraction catheter, the retraction wire further comprising: a plurality of struts (170) coupled to the distal end of the retracting wire at a first end of each of the plurality of struts; a collapsible ring (180) coupled to the second end of each of the plurality of struts; and a collapsible web (190) coupled to the ring and configured to expand into a basket when extended beyond the distal end of the retraction catheter; wherein the collapsible web is configured to fold into a substantially liner structure to movably fit within the retraction catheter.

Example 2 is directed to the thrombectomy device of example 1, wherein the collapsible ring is configured to collapse into a folded state when retracted into the retraction catheter.

Example 3 is directed to the thrombectomy device of example 2, wherein in the folded state the collapsible ring is movable within the retraction catheter.

Example 4 is directed to the thrombectomy device of example 1, wherein the collapsible ring is configured to unfold to assume a substantially circular form when extended beyond the distal end of the retraction catheter.

Example 5 is directed to the thrombectomy device of example 4, wherein unfolding of the collapsible ring directs expansions of the collapsible web beyond the proximal end of the retraction catheter.

Example 6 is directed to the thrombectomy device of example 1, wherein the basket is configured to encircle a clot (110) within the body lumen.

Example 7 is directed to the thrombectomy device of example 1, wherein the plurality of struts (170) are conjointly coupled to the distal end of the retracting wire.

Example 8 is directed to the thrombectomy device of example 1, wherein the collapsible web is configured to fold into a substantially acicular shape to move within the retraction catheter.

Example 9 is directed to the thrombectomy device of example 1, further comprising a secondary catheter (130) for receiving the retraction catheter (150), the secondary catheter having a collection basket (135) at the distal end thereon.

Example 10 is directed to the thrombectomy device of example 9, wherein the collapsible web and the collection basket cooperate to entrap the clot.

Example 11 is directed to the thrombectomy device of example 1, further comprising a guide wire (140) protruding from the collapsible web, the guide wire configured to penetrate through the clot.

Example 12 is directed to the thrombectomy device of example 1, further comprising a retractor at the proximal end of the thrombectomy device to deploy folding and unfolding of the collapsible ring.

Example 13 is directed to the thrombectomy device of example 9, further comprising a tertiary catheter to receive the retraction catheter and the secondary catheter.

Example 14 is directed to the thrombectomy device of example 1, further comprising an Aligner (1120) coupled to the plurality of struts, wherein release of the Aligner retains the collapsible ring in an open position substantially throughout the retraction phase.

Example 15 is directed to the thrombectomy device of example 14, wherein in the open position the Aligner causes the plurality of struts to remain substantially radially expanded within the body lumen.

Example 16 is directed to a method for extracting a clot (110) from a body lumen, the method comprising: percutaneously accessing the body by inserting a guiding catheter (120) into the body lumen; extending a retraction catheter from the guiding catheter into the body lumen and extending the distal end of the retraction catheter through the clot; extending a retraction wire (160) positioned inside the retraction catheter (150) beyond the distal end of the retraction catheter to thereby cause a plurality of struts (170) coupled to the distal end of the retracting wire to unfold a collapsible web (190); retracting the collapsible web to substantially encircle clot; and retracting the encircled clot into the distal end of the retraction catheter.

Example 17 is directed to the method of example 16, wherein retracting the encircled clot into the distal end of the retraction catheter collapses the web into a substantially acicular structure movable within the retraction catheter.

Example 18 is directed to the method of example 16, wherein the collapsible web (190) further comprises a collapsible ring (180).

Example 19 is directed to the method of example 18, wherein the collapsible ring (180) further comprises a plurality of ring segments (384).

Example 20 is directed to the method of example 16, wherein the collapsible ring (180) communicates with the retracting wire (160) through a plurality of struts (170).

Example 21 is directed to the method of example 20, wherein the plurality of struts (170) are conjointly coupled to the distal end of the retracting wire.

Example 22 is directed to the method of example 16, wherein the step of extending a retraction wire beyond the distal end of the retraction catheter further comprises unfolding a collapsible ring (180) to thereby unfold a collapsible web (190).

Example 23 is directed to the method of example 16, wherein the collapsible ring (180) is configured to assume a substantially circular form when deployed.

Example 24 is directed to the method of example 23, wherein unfolding of the collapsible ring directs expansions of the collapsible web beyond the proximal end of the retraction catheter.

Example 25 is directed to the method of example 16, wherein the collapsible web (190) is configured to fold into a substantially acicular shape to move within the retraction catheter.

Example 26 is directed to the method of example 16, further comprising receiving the retraction catheter (150) at a secondary catheter (130), wherein the secondary catheter (130) further comprises a collection basket (135) at the distal end thereon.

Example 27 is directed to the method of example 26, further comprising entrapping the clot at the collection basket (135).

Example 28 is directed to the method of example 16, wherein the step of extending a retraction wire (160) further comprises opening an Aligner (1120) coupled to the plurality of struts (1106).

Example 29 is directed to the method of example 28, wherein the step of opening the Aligner further causes the collapsible ring to remain in an open position substantially throughout the retraction phase.

Example 30 is directed to the method of example 29, wherein in the open position the Aligner causes the plurality of struts to remain substantially radially expanded within the body lumen.

While the principles of the disclosure have been illustrated in relation to the exemplary embodiments shown herein, the principles of the disclosure are no limited thereto and include any modification, variation or permutation thereof.

What is claimed is:

1. A thrombectomy device to extract a thrombus from a body lumen, the device comprising:
a retraction catheter (150) having a proximal end and a distal end, the retraction catheter configured for insertion into the body lumen;
a retracting wire (160) movably positioned inside the retraction catheter, the retraction wire further comprising:
a plurality of struts (170) coupled to the distal end of the retracting wire at a first end of each of the plurality of struts;
a collapsible ring (180) coupled to the second end of each of the plurality of struts; and
a collapsible web (190) coupled to the ring and configured to expand into a basket when extended beyond the distal end of the retraction catheter;
wherein the collapsible web is configured to fold into a substantially liner structure to movably fit within the retraction catheter.

2. The thrombectomy device of claim 1, wherein the collapsible ring is configured to collapse into a folded state when retracted into the retraction catheter.

3. The thrombectomy device of claim 2, wherein in the folded state the collapsible ring is movable within the retraction catheter.

4. The thrombectomy device of claim 1, wherein the collapsible ring is configured to unfold to assume a substantially circular form when extended beyond the distal end of the retraction catheter.

5. The thrombectomy device of claim 4, wherein unfolding of the collapsible ring directs expansions of the collapsible web beyond the proximal end of the retraction catheter.

6. The thrombectomy device of claim 1, wherein the basket is configured to encircle a clot (110) within the body lumen.

7. The thrombectomy device of claim 1, wherein the plurality of struts (170) are conjointly coupled to the distal end of the retracting wire.

8. The thrombectomy device of claim 1, wherein the collapsible web is configured to fold into a substantially acicular shape to move within the retraction catheter.

9. The thrombectomy device of claim 1, further comprising a secondary catheter (130) for receiving the retraction catheter (150), the secondary catheter having a collection basket (135) at the distal end thereon.

10. The thrombectomy device of claim 9, wherein the collapsible web and the collection basket cooperate to entrap the clot.

11. The thrombectomy device of claim 1, further comprising a guide wire (140) protruding from the collapsible web, the guide wire configured to penetrate through the clot.

12. The thrombectomy device of claim 1, further comprising a retractor at the proximal end of the thrombectomy device to deploy folding and unfolding of the collapsible ring.

13. The thrombectomy device of claim 9, further comprising a tertiary catheter to receive the retraction catheter and the secondary catheter.

14. The thrombectomy device of claim 1, further comprising an Aligner (1120) coupled to the plurality of struts, wherein release of the Aligner retains the collapsible ring in an open position substantially throughout the retraction phase.

15. The thrombectomy device of claim 14, wherein in the open position the Aligner causes the plurality of struts to remain substantially radially expanded within the body lumen.

16. A method for extracting a clot (110) from a body lumen, the method comprising:
percutaneously accessing the body by inserting a guiding catheter (120) into the body lumen;
extending a retraction catheter from the guiding catheter into the body lumen and extending the distal end of the retraction catheter through the clot;
extending a retraction wire (160) positioned inside the retraction catheter (150) beyond the distal end of the retraction catheter to thereby cause a plurality of struts (170) coupled to the distal end of the retracting wire to unfold a collapsible web (190);
retracting the collapsible web to substantially encircle clot; and
retracting the encircled clot into the distal end of the retraction catheter.

17. The method of claim 16, wherein retracting the encircled clot into the distal end of the retraction catheter collapses the web into a substantially acicular structure movable within the retraction catheter.

18. The method of claim 16, wherein the collapsible web (190) further comprises a collapsible ring (180).

19. The method of claim 18, wherein the collapsible ring (180) further comprises a plurality of ring segments (384).

20. The method of claim 16, wherein the collapsible ring (180) communicates with the retracting wire (160) through a plurality of struts (170).

21. The method of claim 20, wherein the plurality of struts (170) are conjointly coupled to the distal end of the retracting wire.

22. The method of claim 16, wherein the step of extending a retraction wire beyond the distal end of the retraction catheter further comprises unfolding a collapsible ring (180) to thereby unfold a collapsible web (190).

23. The method of claim 16, wherein the collapsible ring (180) is configured to assume a substantially circular form when deployed.

24. The method of claim 23, wherein unfolding of the collapsible ring directs expansions of the collapsible web beyond the proximal end of the retraction catheter.

25. The method of claim 16, wherein the collapsible web (190) is configured to fold into a substantially acicular shape to move within the retraction catheter.

26. The method of claim 16, further comprising receiving the retraction catheter (150) at a secondary catheter (130), wherein the secondary catheter (130) further comprises a collection basket (135) at the distal end thereon.

27. The method of claim 26, further comprising entrapping the clot at the collection basket (135).

28. The method of claim 16, wherein the step of extending a retraction wire (160) further comprises opening an Aligner (1120) coupled to the plurality of struts (1106).

29. The method of claim 28, wherein the step of opening the Aligner further causes the collapsible ring to remain in an open position substantially throughout the retraction phase.

30. The method of claim 29, wherein in the open position the Aligner causes the plurality of struts to remain substantially radially expanded within the body lumen.
